(19) United States
(12) Patent Application Publication
(10) Pub. No.: US 2014/0057860 A1
(43) Pub. Date: Feb. 27, 2014

(54) PHARMACEUTICAL COMPOSITION FOR TREATING CARDIOVASCULAR AND CEREBROVASCULAR DISEASES AND METHOD OF MANUFACTURING THE SAME

(71) Applicant: Macau University of Science and Technology, Macau (CN)

(72) Inventors: Tao YI, Macau (CN); Liang LI, Macau (CN); Wai Kei Christopher LAM, Macau (CN)

(73) Assignee: Macau University of Science and Technology, Macau (CN)

(21) Appl. No.: 13/972,889

(22) Filed: Aug. 21, 2013

(30) Foreign Application Priority Data
Aug. 27, 2012 (CN) CN 201210308893.7

(51) Int. Cl. A61K 31/7048

(52) U.S. Cl. CPC A61K 31/7048
USPC 514/27

(57) ABSTRACT

The present invention relates to a pharmaceutical composition for treating cardiovascular and cerebrovascular diseases and the method of preparation thereof. The aforesaid pharmaceutical composition is prepared by the following components in weight percent: 0.1%-0.3% scutellarin, 20%-25% co-surfactant, 40-50% surfactant, and 25-30% oil. The method of preparing the aforesaid pharmaceutical composition comprises the steps of: (1) dispersing scutellarin in co-surfactant and surfactant to obtain a mixture; (2) dispersing the mixture from step (1) in oil, and thermostatically and magnetically stirring the dispersion mixture under the temperature of 25° C. to 37° C., such that the components thereof are completely dissolved to obtain the pharmaceutical composition. Through the use of the selected pharmaceutical adjuvants having the ability to inhibit MRP2, the aforesaid pharmaceutical composition effectively improves absorption and bioavailability of scutellarin. The preparation of the aforesaid pharmaceutical composition is simple and convenient, and the pharmaceutical composition can be processed into a variety of dosage forms for oral administration.

### CROSS-REFERENCE TO RELATED APPLICATION

This application claims benefit under 35 U.S.C. §119(e) of Chinese Application having Serial No. CN 201210308893.7 filed 27 Aug., 2012, which is hereby incorporated by reference herein in its entirety.

### FIELD OF INVENTION

This invention relates to a pharmaceutical composition for treating cardiovascular and cerebrovascular diseases and a method of preparation thereof. In particular, it relates to a pharmaceutical composition for treating cardiovascular and cerebrovascular diseases comprising scutellarin and a method of preparation thereof, wherein the pharmaceutical composition makes use of the inhibition effect on transport protein to enhance absorption.

### BACKGROUND OF INVENTION

Breviscapine is the flavonoid extracted from the dried whole plant of Erigeron breviscapus (Vant.) Hand Mazz. Scutellarin, chemically known as 4,5,6-trihydroxyflavone-7-glucuronide, accounting for more than 90% of breviscapine. Recently, scutellarin has been used for treating cardiovascular and cerebrovascular diseases, and demonstrated from research for neuron protection, prevention of cytotoxicity, regulation of vascular endothelial function, alleviation of cerebral vasospasm, improvement on micro-circulation, hyperlipidemia reduction, immunity regulation, alleviation on inflammation responses, protection against free radical damage, and inhibition of platelet aggregation, etc. From the 60s to the 90s of the 20^{th} century, injections, tablets and granule forms have been clinically developed, but there is still room for improvement on the bioavailability of these pharmaceuticals. There are good market prospects with high economic values for the use of scutellarin for treatment and prevention of cardiovascular and cerebrovascular diseases.

A number of patent publications has been found from the Chinese patent database and the majority of the existing patents/patent publications relates to methods for preparing different formulations of breviscapine:

Chinese patent publication CN1875981 (Publication No.) describes an invention by Gao, Chun-Ping entitled “An injectable emulsion containing Breviscapine and a method of preparing the same”, involving a freeze-dried injectable emulsion containing Breviscapine and a method of preparing the same.

Chinese patent publication CN1593449 (Publication No.) describes an invention by Wang, Bing et al entitled “A self-emulsifying soft capsule containing Breviscapine and a method of preparing the same”, involving an optimized formulation of the self-emulsifying soft capsule containing Breviscapine and a method of preparing the same.

Chinese patent publication CN1383817 (Publication No.) describes an invention by Zhang, Jun-Shou et al entitled “An oral formulation of a sustained release agent containing Breviscapine”, in which the oral formulation can prolong duration of the action of the drug.

Chinese patent publication CN1596905 (Publication No.) describes an invention by Xie, Ya-Su entitled “A dispersible tablet of Breviscapine”, in which the dispersible tablet is suitable for a special group of patients.

Chinese patent publication CN101336886 (Publication No.) describes an invention by Wu, Zheng-Hong et al entitled “A soluble formulation of Breviscapine”, involving the use of dendrimers to enhance solubility of Breviscapine.

Chinese patent publication CN102048691A (Publication No.) describes an invention by Tang, Yong et al entitled “An oral spray containing Breviscapine and a method of preparing the same”, in which the oral spray triggers a rapid onset and a method of preparing the same is disclosed.

Chinese patent publication CN101543481 (Publication No.) describes an invention by Zhang, Jian-Li entitled “A double-layer sustained release tablet containing Breviscapine and a method of preparing the same”, in which the tablet has a layer for immediate release and a layer for sustained release, and a method of preparing the same is disclosed.

Chinese patent publication CN101439041 (Publication No.) describes an invention by Li, Yong-qiang et al entitled “A Chinese medicine granule containing Breviscapine and a method of preparing the same”, in which the granule is processed into a ball shaped granule and a method of preparing the same is disclosed.

Chinese patent publication CN101088505 (Publication No.) describes an invention by Wang, Yi-ming et al entitled “A polymeric nano-preparation of Breviscapine and a method of preparing the same”, in which the preparation is made into a nano granules and a method of preparing the same is disclosed.

Chinese patent publication CN1965848 (Publication No.) describes an invention by Ren, Yang-Fan entitled “A freeze-dried powder for injection of Breviscapine and a method of preparing the same”, in which the powder for injection possesses a relatively high content of drug and a method of preparing the same is disclosed.

Chinese patent publication CN1939320 (Publication No.) describes an invention by Weng, Wei-Yu et al entitled “An effervescent dry suspension of Breviscapine and a method of preparing the same”, in which the suspension has a high dispersing power and a method of preparing the same is disclosed.

Chinese patent publication CN1843368 (Publication No.) describes an invention by Luo, Guo-An et al entitled “A long-circulating nanoliposome of Breviscapine and a method of preparing the same”, in which the particle size of the nanoliposome is uniform and a method of preparing the same is disclosed.

Chinese patent publication CN1830451 (Publication No.) describes an invention by Zhang, Yu-Mei entitled “A glucose injection of Breviscapine and a method of preparing the same”, involving the method of preparing a glucose injection with good stability.

### SUMMARY OF INVENTION

In vivo bioavailability of scutellarin is poor, not only due to the poor water solubility of scutellarin, but more importantly, transport proteins on small intestine such as human multidrug resistance-associated protein 2 (MRP2) promotes the efflux of drugs, resulting in a decreased absorption of scutellarin. As such, clinical usage and popularity of scutellarin agents has been greatly limited. MRP2 is distributed in liver, small intestine, kidney and apical membrane of brain tissue, with large differences in expression levels in these areas. In addition, MRP2, being a glutathione-dependent efflux pump, has multiple binding sites and a high selectivity of inhibitors. Studies have revealed that there are many types of pharmaceutical adjuvants that can inhibit MRP2 in human small intestinal epithelial cells to promote drug absorption. It has been reported in literatures that there are many ways in screening drugs or adjuvants for inhibiting MRP2, among which the most important is the use of human colon epithelial cells (Caco-2) model test and insect Sf9 overexpressing MRP2 membrane vesicles transport test, while a variety of pharmaceutical adjuvants such as polyethylene glycol etc. have been reported to inhibit MRP2 on the small intestinal epithelial cells to promote drug absorption.

In the light of the foregoing background, it is an object of the present invention to provide an alternate pharmaceutical composition for treating cardiovascular and cerebrovascular diseases in which this pharmaceutical composition inhibits the efflux of scutellarin by multidrug resistance-associated protein 2 (MRP2) on small intestinal epithelial cells so as to enhance absorption and bioavailability of scutellarin.

Accordingly, a technical solution of the present invention for achieving the aforesaid object, in one aspect, is:

A pharmaceutical composition for treating cardiovascular and cerebrovascular diseases comprising 20-25 weight percent co-surfactant, 40-50 weight percent surfactant, 25-30 weight percent oil, and 0.1-0.3 weight percent scutellarin, wherein the weight percentages of the above components add up to 100%.

In an exemplary embodiment of the present invention, the co-surfactant is diethylene glycol monoethylether.

In another exemplary embodiment, the surfactant is selected from a group consisting of polyoxyethylene castor oil, polyoxyethylene hydrogenated castor oil, and gaprylocaproyl macrogolglycerides, and any combinations thereof.

In yet another exemplary embodiment, the oil is selected from a group consisting of glyceryl monolinoleate, Capmul® MCM, and the combinations thereof.

According to another aspect of the present invention, a method of preparing a pharmaceutical composition for treating cardiovascular and cerebrovascular diseases is provided in which the method is simple and convenient.

Accordingly, a technical solution of the present invention for achieving the aforesaid object of this aspect is:

A method of preparing a pharmaceutical composition for treating cardiovascular and cerebrovascular diseases, comprising the steps of:

a) dispersing scutellarin in co-surfactant and surfactant to obtain a mixture; and

b) dispersing the mixture from step (a) in oil, and thermostatically stirring the dispersion mixture under 25° C. to 37° C., such that the components thereof are completely dissolved to obtain the pharmaceutical composition.

In another aspect of the present invention, a use of a pharmaceutical composition for treating cardiovascular and cerebrovascular diseases is demonstrated, and a technical solution for achieving this object of this aspect is:

In another exemplary embodiment, the use of a pharmaceutical composition for treating cardiovascular and cerebrovascular diseases is provided in which through the Caco-2 model experiment and insect Sf9 overexpressing MRP2 membrane vesicles transport test, diethylene glycol monoethylether, polyoxyethylene castor oil, polyoxyethylene hydrogenated castor oil, gaprylocaproyl macrogolglycerides, glyceryl monolinoleate and Capmul® MCM of the pharmaceutical composition are shown to inhibit MRP2 on human small intestine and thus play an important role in promoting the scutellarin absorption.

In another exemplary embodiment of the use of a pharmaceutical composition for treating cardiovascular and cerebrovascular diseases, the pharmaceutical composition comprising scutellarin is processed into an oral microemulsion or a solid self-microemulsion of a sustained or controlled release agent, which is used for the treatment of cardiovascular and cerebrovascular diseases.

In yet another exemplary embodiment of the use of a pharmaceutical composition for treating cardiovascular and cerebrovascular diseases, the pharmaceutical composition of scutellarin efficiently increases bioavailability of scutellarin, while the method of preparation thereof is simple.

In another aspect of the present invention, a pharmaceutical composition for treating cardiovascular and cerebrovascular diseases is provided that comprises scutellarin and an adjuvant selected from a group consisting of a co-surfactant, a surfactant, an oil, a solid carrier, and any combinations thereof. The pharmaceutical composition reduces the efflux of scutellarin and enhances absorption of scutellarin by inhibiting MRP2 on small intestinal epithelial cells.

In one exemplary embodiment, the co-surfactant is selected from a group consisting of diethylene glycol monoethylether, polyethylene glycol (PEG), and any combinations thereof. In a further exemplary embodiment, diethylene glycol monoethylether is Transcutol®; PEG is PEG 400 or PEG 2000.

In one exemplary embodiment, the surfactant is selected from a group consisting of polyoxyethyleneglycerol triricinoleate 35 castor oil, polyoxyethylene hydrogenated castor oil, poloxamer 407, poloxamer 188, gaprylocaproyl macrogolglycerides, and any combinations thereof. In a further exemplary embodiment, polyoxyethyleneglycerol triricinoleate 35 castor oil is Cremophor® EL; polyoxyethylene hydrogenated castor oil is Cremophor® RH; poloxamer 407 is Pluronic® F127; poloxamer 188 is Pluronic® F68; gaprylocaproyl macrogolglycerides is Labrasol®.

In one exemplary embodiment, the oil is selected from a group consisting of glyceryl monolinoleate, medium chain triglycerides, C8/C10 mono-/di-glycerides, and any combinations thereof. In a further exemplary embodiment, glyceryl monolinoleate is Maisine® 35-1; medium chain triglycerides is Labrafac Lipophile® WL 1349; C8/C10 mono-/di-glycerides is Capmul® MCM.

In one exemplary embodiment, the solid carrier is β-cyclodextrin. In a further exemplary embodiment, the solid carrier is lactose, hydroxypropyl methyl cellulose (HPMC) K4M, or hydroxypropyl methyl cellulose K100.

In yet a further exemplary embodiment, the pharmaceutical composition of consists of scutellarin and polyoxyethyleneglycerol triricinoleate 35 castor oil. The pharmaceutical composition of this specific embodiment can further reduce the efflux of scutellarin and enhance absorption of scutellarin by inhibiting MRP2 on small intestinal epithelial cells. In a further exemplary embodiment, the pharmaceutical composition further comprises poloxamer 407, PEG 2000, β-cyclodextrin, and any combinations thereof.

A further aspect of the present invention provides a pharmaceutical composition for treating cardiovascular and cerebrovascular diseases comprising scutellarin and polyoxyethyleneglycerol triricinoleate 35 castor oil. In one exemplary embodiment, the pharmaceutical composition further comprises poloxamer 407, PEG 2000, and any combinations thereof.

A further aspect of the present invention provides a method of treating cardiovascular and cerebrovascular diseases in a human patient comprising administering to the patient a pharmaceutical composition comprising scutellarin, polyoxyethyleneglycerol triricinoleate 35 castor oil, and an adjuvant wherein the adjuvant exhibits a synergistic effect with polyoxyethyleneglycerol triricinoleate 35 castor oil in treating cardiovascular and cerebrovascular diseases. In an exemplary embodiment, the adjuvant is selected from a group consisting of poloxamer 407 and PEG 2000.

The present invention utilizes the inhibitory action of pharmaceutical adjuvants of a pharmaceutical composition for treating cardiovascular and cerebrovascular diseases (hereinafter also referred to a pharmaceutical composition of scutellarin) on MRP2 on small intestinal epithelial cells; through the study on the selection of the selected pharmaceutical adjuvants on the inhibitory action on MRP2 and the technique in the preparation thereof, a pharmaceutical composition of scutellarin is obtained. The combination of the study on the preparation and the clinical applications of the pharmaceutical composition provides the basic research data for the discovery of the pharmaceutical composition of scutellarin with the best absorption effect of scutellarin.

In the present invention, through the Caco-2 model test and insect Sf9 overexpressing MRP2 membrane vesicles transport test, a pharmaceutical composition comprising scutellarin and any one of the pharmaceutical adjuvants of diethylene glycol monoethylether, polyoxyethylene castor oil, polyoxyethylene hydrogenated castor oil, gaprylocaproyl macrogolglycerides, glyceryl monolinoleate, Capmul® MCM is proven to inhibit MRP2 on human small intestine and thus plays an important role in promoting the scutellarin absorption. The pharmaceutical composition of scutellarin is processed into an oral microemulsion, or a solid self-microemulsion of a sustained or controlled release agent, which is used for the treatment of cardiovascular and cerebrovascular diseases. The pharmaceutical composition for treating cardiovascular and cerebrovascular diseases of the present invention resolves the technical problem of the efflux of scutellarin by MRP2 on small intestinal epithelial cells. As a result, absorption of scutellarin is enhanced and bioavailability of scutellarin is efficiently increased. Further, the method of preparation of the pharmaceutical composition is simple.

In short, advantages of the pharmaceutical composition for treating cardiovascular and cerebrovascular diseases of the present invention include, but are not limited to, enhanced absorption of scutellarin and increased bioavailability of scutellarin, whereas the method of preparation thereof is simple.

### BRIEF DESCRIPTION OF FIGURES

FIG. 1 shows the results of a study on the efflux ratio of scutellarin with 20 μM inhibitor (MK571) and scutellarin with 15 excipients in Caco-2 monolayers (* denotes p<0.05).

FIG. 2 shows the results of a study on vesicles transport assay of MRP2 inhibition by scutellarin and excipients according to one embodiment of the present invention (* denotes p<0.05).

FIG. 3 shows the results of a study on the synergistic effect of 9 excipients with Cremophor® EL in Caco-2 model according to one embodiment of the present invention (*denotes p<0.05; 1: Cremophor EL, 2: Cremophor EL+ Cremophor RH, 3: Cremophor EL+ Labrasol, 4: Cremophor EL+ Pluronic F68, 5: Cremophor EL+ Pluronic F127, 6: Cremophor EL+ PEG 2000, 7: Cremophor EL+ PEG 400, 8: Cremophor EL+ Transcutol, 9: Cremophor EL+ Maisine 35-1, 10: Cremophor EL+ β-cyclodextrin)

FIG. 4 shows the results of a study on the synergistic inhibition effect of 5 excipients with Cremophor® EL in MRP2 transport model according to one embodiment of the present invention (* denotes p<0.05).

### DETAILED DESCRIPTION OF THE PREFERRED EMBODIMENTS

As used herein and in the claims, “comprising” means including the following elements but not excluding others.

Caco-2 cells are epithelial cells from human colorectal carcinoma, containing enzymes related to small intestinal brush border epithelium such as P-glycoprotein and multidrug resistance-associated protein, etc. Caco-2 cells grown on a porous and permeable polycarbonate membrane can serve as a small intestinal absorption model for screening drugs or pharmaceutical adjuvants for inhibition of the transporter protein. Through the selection of MRP2 inhibitor MK571, scutellarin is identified as the substrate for MRP2. Thus, based on the transport study of the Caco-2 model, the efflux ratio of the individual mixture of scutellarin with diethylene glycol monoethylether, polyoxyethylene castor oil, polyoxyethylene hydrogenated castor oil, gaprylocaproyl macrogolglycerides, glyceryl monolinoleate, and Capmul® MCM can be obtained from the forward and reverse transport volume between the apical (AP) side to the basolateral (BL) side. On comparing the results from study of scutellarin without the aforesaid pharmaceutical adjuvants, the selected pharmaceutical adjuvants can thus be proven to exhibit inhibitory action on MRP2 transporter protein to promote intestinal absorption.

In MRP2 membrane vesicle transport test, insect Sf9 membrane vesicles overexpressing MRP2 are used. Since the main function carried out by MRP2 transport protein is unidirectional, ATP is transported by consumption of ATP to transport amphoteric anionic compounds. It is difficult to flexibly adjust the concentration of ATP in intact cells and thus, there are many advantages in studying the specificity of MRP2 substrate and in screening MRP2 inhibitors. MRP2 membrane vesicles transport test is divided into two parts; ATP enzyme activities of diethylene glycol monoethylether, polyoxyethylene castor oil, polyoxyethylene hydrogenated castor oil, gaprylocaproyl macrogolglycerides, glyceryl monolinoleate, and Capmul® MCM are first determined by overexpressed MRP2 membranes, and then through the inhibition test on overexpressed MRP2 vesicles, inhibitory action of scutellarin on transporter protein was compared.

A pharmaceutical composition comprising scutellarin is processed into an oral microemulsion or a solid self-microemulsion for the treatment of cardiovascular and cerebrovascular diseases.

The present invention is further explained by the following examples.

### Example 1

A pharmaceutical composition for treating cardiovascular and cerebrovascular diseases is prepared by the following components in weight percent: 24% co-surfactant, 47.7% surfactant, 28% oil, and 0.3% drug.

The drug is scutellarin.

The co-surfactant is diethylene glycol monoethylether.

The surfactant is a mixture of polyoxyethylene castor oil and gaprylocaproyl macrogolglycerides (in 1:1 weight ratio).

The oil is a mixture of glyceryl monolinoleate and Capmul® MCM (Abitec, USA) (in 1:1 weight ratio).

### Method of Preparation

Weighed co-surfactant and surfactant of diethylene glycol monoethylether, polyoxyethylene castor oil and gaprylocaproyl macrogolglycerides were first well mixed, scutellarin was dispersed therein, and then the well-mixed oil mixture of glyceryl monolinoleate and Capmul® MCM was slowly added thereto. The mixture was thermostatically and magnetically stirred under 25° C. such that the components thereof were completely dissolved to obtain the pharmaceutical composition of scutellarin.

### Example 2

A pharmaceutical composition for treating cardiovascular and cerebrovascular diseases is prepared by the following components in weight percent: 23.6% co-surfactant, 46.3% surfactant, 30% oil, and 0.1% drug.

The drug is scutellarin.

The co-surfactant is diethylene glycol monoethylether.

The surfactant is a mixture of polyoxyethylene hydrogenated castor oil and gaprylocaproyl macrogolglycerides (in 4:1 weight ratio).

The oil is a mixture of glyceryl monolinoleate and Capmul® MCM (in 2:1 weight ratio).

### Method of Preparation

Scutellarin was first dispersed in the well-mixed oil of glyceryl monolinoleate and Capmul® MCM, and well-mixed co-surfactant and surfactant mixture of diethylene glycol monoethylether, polyoxyethylene hydrogenated castor oil and gaprylocaproyl macrogolglycerides was slowly added. The mixture was thermostatically and magnetically stirred under 37° C. such that the components thereof were completely dissolved to obtain the pharmaceutical composition of scutellarin.

### Example 3

A pharmaceutical composition for treating cardiovascular and cerebrovascular diseases is prepared by the following components in weight percent: 20% co-surfactant, 49.9% surfactant, 30% oil, and 0.1% drug.

The drug is scutellarin.

The co-surfactant is diethylene glycol monoethylether.

The surfactant is a mixture of polyoxyethylene castor oil, polyoxyethylene hydrogenated castor, and gaprylocaproyl macrogolglycerides (in 1:1:1 weight ratio).

The oil is glyceryl monolinoleate.

### Method of Preparation

Scutellarin was first dispersed in glyceryl monolinoleate, and well-mixed co-surfactant and surfactant mixture of diethylene glycol monoethylether, polyoxyethylene castor oil, polyoxyethylene hydrogenated castor, and gaprylocaproyl macrogolglycerides was slowly added. The mixture was thermostatically and magnetically stirred under 37° C. such that the components thereof were completely dissolved to obtain the pharmaceutical composition of scutellarin.

### Example 4

A pharmaceutical composition for treating cardiovascular and cerebrovascular diseases is prepared by the following components in weight percent: 25% co-surfactant, 45% surfactant, 29.8% oil, and 0.2% drug.

The drug is scutellarin.

The co-surfactant is diethylene glycol monoethylether.

The surfactant is polyoxyethylene castor oil.

The oil is Capmul® MCM.

### Method of Preparation

Scutellarin was first dispersed in Capmul® MCM, and well-mixed co-surfactant and surfactant mixture of diethylene glycol monoethylether and polyoxyethylene castor oil was slowly added. The mixture was thermostatically and magnetically stirred under 25° C. such that the components thereof were completely dissolved to obtain the pharmaceutical composition of scutellarin.

### Example 5

A pharmaceutical composition for treating cardiovascular and cerebrovascular diseases is prepared by the following components in weight percent: 21% co-surfactant, 49.7% surfactant, 29% oil, and 0.3% drug.

The drug is scutellarin.

The co-surfactant is diethylene glycol monoethylether.

The surfactant is gaprylocaproyl macrogolglycerides.

The oil is a mixture of glyceryl monolinoleate and Capmul® MCM (in 1:3 weight ratio).

### Method of Preparation

Scutellarin was first dispersed in the well-mixed oil of glyceryl monolinoleate and Capmul® MCM, and well-mixed co-surfactant and surfactant mixture of diethylene glycol monoethylether and gaprylocaproyl macrogolglycerides was slowly added into the dispersion mixture. The mixture was thermostatically and magnetically stirred under 25° C. such that the components thereof were completely dissolved to obtain the pharmaceutical composition of scutellarin.

### Example 6

A pharmaceutical composition for treating cardiovascular and cerebrovascular diseases is prepared by the following components in weight percent: 22.3% co-surfactant, 50% surfactant, 27.5% oil, and 0.2% drug.

The drug is scutellarin.

The co-surfactant is diethylene glycol monoethylether.

The surfactant is a mixture of polyoxyethylene castor oil and polyoxyethylene hydrogenated castor (in 1:1 weight ratio).

The oil is glyceryl monolinoleate.

### Method of Preparation

Scutellarin was first dispersed in glyceryl monolinoleate, and well-mixed co-surfactant and surfactant mixture of diethylene glycol monoethylether, polyoxyethylene castor oil, and polyoxyethylene hydrogenated castor was slowly added. The mixture was thermostatically and magnetically stirred under 30° C. such that the components thereof were completely dissolved to obtain the pharmaceutical composition of scutellarin.

### Example 7

A pharmaceutical composition for treating cardiovascular and cerebrovascular diseases is prepared by the following components in weight percent: 20% co-surfactant, 49.9% surfactant, 30% oil, and 0.1% drug.

The drug is scutellarin.

The co-surfactant is diethylene glycol monoethylether.

The surfactant is gaprylocaproyl macrogolglycerides.

The oil is Capmul® MCM.

### Method of Preparation

Scutellarin was first dispersed in Capmul® MCM, and well-mixed co-surfactant and surfactant mixture of diethylene glycol monoethylether and gaprylocaproyl macrogolglycerides was slowly added. The mixture was thermostatically and magnetically stirred under 27° C. such that the components thereof were completely dissolved to obtain the pharmaceutical composition of scutellarin.

It is necessary to explain a technical problem arisen during the preparation of a pharmaceutical composition of scutellarin. Since scutellarin is poorly soluble in water and chemically unstable, and owing to the fact that a glucose molecule is prone to be removed from scutellarin to form scutellarin aglycone flavonoid, the preparation of pharmaceutical composition of scutellarin should be controlled within a temperature range of 25° C.-37° C. The solubility of scutellarin in each type of oils and surfactants is different and so in preparing pharmaceutical composition of scutellarin in different proportions of oils and surfactants, one skilled in the art may carry out the magnetic stirring in a temperature range with certain differences and variations based on common knowledge in the art.

### Example 8

50 μM scutellarin monomer was separately mixed with the same concentration of diethylene glycol monoethylether, polyoxyethylene castor oil, polyoxyethylene hydrogenated castor oil, gaprylocaproyl macrogolglycerides, glyceryl monolinoleate, and Capmul® MCM. 400 μL sample and 600 μL blank buffer were respectively added to the AP side and the BL side of the Caco-2 cell model that was cultured on porous and permeable polycarbonate membrane for 21 days. Afterwards, 400 μL blank buffer and 600 μL sample were respectively added to the AP side and the BL side of the Caco-2 cell model. The apparent permeability coefficient (Papp) obtained from the scutellarin drawn from the AP and BL sides was used to calculate the efflux ratios. The results are shown in Table 1, showing that there was a significant decrease in the efflux ratios of the six mixtures, each mixture containing one of the six pharmaceutical adjuvants and scutellarin, as compared to the efflux ratio of the scutellarin monomer with the same concentration. This result proved that the selected pharmaceutical adjuvants of the pharmaceutical composition can be used to promote scutellarin absorption, which further demonstrated the inhibitory action of diethylene glycol monoethylether, polyoxyethylene castor oil, polyoxyethylene hydrogenated castor oil, gaprylocaproyl macrogolglycerides, glyceryl monolinoleate, and Capmul® MCM on MRP2.

In the insect Sf9 overexpressing MRP2 membrane test for studying ATP enzyme activity on 96-well plates, E2-17βG was selected as the MRP2 substrate in the sample well according to the phosphate standard curve from the standard well. In this test, 50 μM scutellarin monomer was separately mixed with the same concentration of diethylene glycol monoethylether, polyoxyethylene castor oil, polyoxyethylene hydrogenated castor oil, gaprylocaproyl macrogolglycerides, glyceryl monolinoleate, and Capmul® MCM. On comparing with the sodium vanadate control well, the agonist effect of the ATP activity of scutellarin monomers and the six mixtures, each mixture containing one of the six pharmaceutical adjuvants and scutellarin, were shown in Table 1.

In the insect Sf9 overexpressing MRP2 vesicle test for studying inhibitory effect on 96-well plates, carboxylic acid was used as a positive control solution. Concentrations of 5 μM, 10 μM, 25 μM, 50 μM were selected near the 1050 median inhibitory concentration of the six pharmaceutical adjuvants, and MgATP and MgAMP were respectively added into two sample wells in which each of the sample wells contained the same sample. Upon reaction at 37° C. for 40 min, 0.2 ml stop buffer was added to terminate the reaction and the mixture was then quickly transferred to a 96-well filter plate, on which it was quickly filtered by 0.7 μm glass fiber filter. ATP-dependent transport was identified as the difference in the scutellarin content of the two transports. The results were shown in Table 1.

The above experiments proved that the pharmaceutical adjuvants of diethylene glycol monoethylether, polyoxyethylene castor oil, polyoxyethylene hydrogenated castor oil, gaprylocaproyl macrogolglycerides, glyceryl monolinoleate, and Capmul® MCM were exhibited the inhibitory function on MRP2 on human small intestine, which in turn enhanced oral absorption of the drug of the pharmaceutical composition.

TABLE 1
Results of Example 8 on study of the inhibitory action of Caco-2
efflux ratio and MRP2 membrane vesicle transport

| | | ATP | |
|---|---|---|---|
| | | enzymatic | Inhibitory |
| | | activity | action of |
| | Caco-2 | of MRP2 | MRP2 |
| | Efflux | membrane | vesicle |
| Sample | Ratio (%) | (nM/L) | (μg) |
| Scutellarin | 5.2390 | 11.749 | 573.85 |
| Scutellarin + Polyoxyethylene | 1.2075 | 14.978 | 1038.4 |
| castor oil | | | |
| Scutellarin + Gaprylocaproyl | 2.5023 | 7.425 | 588.64 |
| macrogolglycerides | | | |
| Scutellarin + Diethylene | 2.2233 | 10.530 | 639.46 |
| glycol monoethylether | | | |
| Scutellarin + Polyoxyethylene | 1.7338 | 11.879 | 685.54 |
| hydrogenated castor oil | | | |
| Scutellarin + Glyceryl | 1.2384 | 5.146 | 390.23 |
| monolinoleate | | | |
| Scutellarin + Capmul ® MCM | 1.6709 | 4.883 | 379.95 |

### Example 9

A pharmaceutical composition for treating cardiovascular and cerebrovascular diseases for the preparation of an oral microemulsion or a solid self-microemulsion of a sustained release agent is used for the treatment of cardiovascular and cerebrovascular diseases, in which scutellarin acts as the active ingredient in the pharmaceutical composition. Scutellarin was first dispersed in the well-mixed oil of glyceryl monolinoleate (Maisine® 35-1) and Capmul® MCM, and well-mixed co-surfactant and surfactant mixture of Transcutol®, Cremophor® E1, and Pluronic® F127 was slowly added into the dispersion mixture (with the same composition of Example 1). The mixture was thermostatically and magnetically stirred at 37° C. such that the components thereof were completely dissolved to obtain the pharmaceutical composition of scutellarin. This pharmaceutical composition can be directly processed into an oral microemulsion in which the particle size of the micro-emulsion upon water re-dispersion is less than 100 nm. From the accelerated test of storing the pharmaceutical composition for 3 months under temperature of 60° C. and relative humidity of 75% and the long-term stability test performed under temperature 25° C., the content of scutellarin in the pharmaceutical composition of scutellarin was proven to be stable, in which the relative standard deviation (RSD) of the content change is less than 5%.

On comparing with ordinary tablets, the oral microemulsion prepared from a pharmaceutical composition for treating cardiovascular and cerebrovascular diseases has a better dispersion state with rapid drug dissolution. In simulated intestinal fluid, drug dissolution percentage was over 80% within 5 minutes. The oral microemulsion can be dispersed in water for administration which would be suitable for elderly, and patients suffering from stroke and dysphagia. For patients suffering from acute attack of coronary heart disease or angina pectoris, rapid onset can be triggered upon administration of the oral microemulsion to effectively control the disease.

### Example 10

A solid self-microemulsion of a sustained release agent prepared from a pharmaceutical composition for treating cardiovascular and cerebrovascular diseases can also be used to treat cardiovascular disease. Scutellarin was first dispersed in the well-mixed oil of glyceryl monolinoleate (Maisine® 35-1) and Capmul® MCM, and well-mixed co-surfactant and surfactant mixture of Transcutol®, Cremophor® E1, and Pluronic® F127 was slowly added into the dispersion mixture (with the same composition of Example 1). The mixture was thermostatically and magnetically stirred at 37° C. such that the components thereof were completely dissolved to obtain the pharmaceutical composition of scutellarin. Afterwards, an appropriate amount of water and β-cyclodextrin was added into and mixed with the pharmaceutical composition of scutellarin. Upon spray drying or freeze drying, the pharmaceutical composition of scutellarin can be further processed into a solid self-microemulsion having sustained or controlled release property. From the accelerated test of storing the pharmaceutical composition for 3 months under temperature of 60° C. and relative humidity of 75% and the long-term stability test performed under temperature 25° C., the content of scutellarin in the pharmaceutical compositions of scutellarin was proven to be stable, in which the relative standard deviation (RSD) of the content change is less than 5%.

A solid self-microemulsion of a sustained release agent prepared from a pharmaceutical composition for treating cardiovascular and cerebrovascular diseases, through the controlled release of drug by scutellarin, can gradually reduce hemorheology indices such as high-shear whole blood viscosity, low-shear whole blood viscosity, plasma viscosity, blood reduced viscosity, erythrocyte aggregation index, erythrocyte deformation index, and platelet count, resulting in reduction in blood viscosity, increase in red blood cell deformability, inhibiting aggregation of platelet and red blood cells, and improving microcirculation. In addition, cholesterol and triglycerides are significantly lowered to prevent atherosclerosis, which is conducive to microvascular perfusion and prevents cerebral ischemia reperfusion induced neuronal apoptosis.

### Example 11

Oral bioavailability of scutellarin monomer is low. Scutellarin was first dispersed in the well-mixed oil of glyceryl monolinoleate (Maisine® 35-1) and Capmul® MCM, and well-mixed co-surfactant and surfactant mixture of Transcutol®, Cremophor® E1, and Pluronic® F127 was slowly added into the dispersion mixture (with the same composition of Example 1). The mixture was thermostatically and magnetically stirred at 30° C. such that the components thereof were completely dissolved to obtain the pharmaceutical composition of scutellarin. This oral pharmaceutical composition of scutellarin was administered to rats at a dose of 100 mg/kg; on comparing with the administration of breviscapine tablets at the same dosage, plasma concentration was significantly improved and bioavailability was increased by two folds. Further, pharmacokinetic parameters also showed that the peak concentration of the pharmaceutical composition of scutellarin and the area under curve (AUC) of the plasma concentration—time curve were significantly improved as compared with breviscapine tablet group (see Table 2), indicating that the pharmaceutical composition for treating cardiovascular disease can improve bioavailability of scutellarin as compared to breviscapine tablets.

TABLE 2
Pharmacokinetic parameters of Example 11 upon administration
of oral pharmaceutical composition of scutellarin at a dose of 100
mg/kg and breviscapine tablets at the same dosage in rats

| | Peak | | | |
|---|---|---|---|---|
| | Concen- | Peak | | |
| | tration | Time | AUC₍₀₋ₜ₎ | AUC_{(0-infinity)} |
| Prescription | (ng/mL) | (h) | (ng/mL * h) | (ng/mL * h) |
| Breviscapine | 197 ± 64 | 4.6 ± 2.6 | 2161.0 ± 910.1 | 2783.2 ± |
| tablets | | | | 1279.5 |
| Pharmaceutical | 285 ± 71 | 3.0 ± 2.7 | 4582.7 ± 834.1 | 7122.3 ± |
| composition of | | | | 1515.2 |
| scutellarin | | | | |

It is necessary to explain a technical problem arisen during the course of the present invention. The present invention relates to an oral microemulsion and a solid self-microemulsion of a sustained release agent prepared from a pharmaceutical composition for treating cardiovascular and cerebrovascular diseases, in which the dosage of the active ingredients thereof are affected by many factors. For instance, the form prepared in the treatment of acute or chronic cardiovascular and cerebrovascular diseases are different due to the different usages in treating these two diseases. Both forms are proven to be relatively stable from the stability test and they are both convenient for storage and transport. Since the activity of scutellarin in both the prepared microemulsion and solid self-microemulsion of the sustained release agent is relatively high, while the particle size thereof is relatively small, the drug is relatively dispersed therein. In addition, the efflux of scutellarin by MRP2 on small intestine is inhibited and so the absorption of scutellarin is enhanced; thus the bioavailability of the pharmaceutical composition of scutellarin is increased as compared to breviscapine tablets.

### Example 12

Caco-2 cells were seeded on 24-well Millicell insert filters with a density of about 1×10⁵ cells/well. These cells were cultured for 21 days to reach confluence and differentiation. The integrity of Caco-2 cell monolayers was evaluated by monitoring trans-epithelial electrical resistance (TEER) on an epithelial volt ohmmeter (World Precision Instruments, Sarasota, Fla., USA). The impermeability (gate functions) of epithelial cell monolayers was measured by the fluorescein leakage test (FLT) that provided information on the effects of xenobiotics on the impermeability of epithelial cell monolayers. The differentiation was assessed using the alkaline phosphatase (ALP) assay at 562 nm

After the above measured parameters of the Caco-2 model became stable, transport study was performed from the apical (AP) to the basolateral (BL) side and from BL to AP at 37° C. Following 30 min of equilibration after the addition of 400 μl HBSS (pH 7.4) in AP side and 600 μl in BL side in an atmosphere of 5% CO₂, the test solutions containing 100 μM scutellarin or each of 100 μg/ml of five surfactants Cremophor® EL, Cremophor® RH, Labrasol®, Pluronic® F68, and Pluronic® F127; each of 100 μg/ml of three oils Labrafac Lipophile® WL1349, Maisine® 35-1, and Capmul® MCM; each of 100 μg/ml of three co-surfactants Transcutol®, PEG 400, and PEG 2000; and each of 100 μg/ml of four solid carriers β-cyclodextrin, lactose, HPMC K4M®, HPMC K100®. The solutions were all dissolved in DMSO and HBSS and then added to the donor side. The sample of 100 μM scutellarin was used as the scutellarin standard group in comparing the excipients efflux effect. Permeability of scutellarin was measured at 37° C. from the AP to BL direction and BL to AP direction after incubating in 5% CO₂ for respectively 30 min, 60 min, and 90 min. At 30 min and 60 min, 100 μl samples were drawn from the BL side when apparent permeability coefficient (Papp) was obtained in the AP to BL direction, and vice versa. After 90 min of incubation, solutions from both AP and BL sides were collected. Then the separately collected samples from each well of AP and BL sides were diluted with equal amounts of methanol before they were injected into the LC-MS for quantification of scutellarin. At 30, 60, 90 min of incubation, TEER values were respectively detected before the samples were drawn from the tested wells.

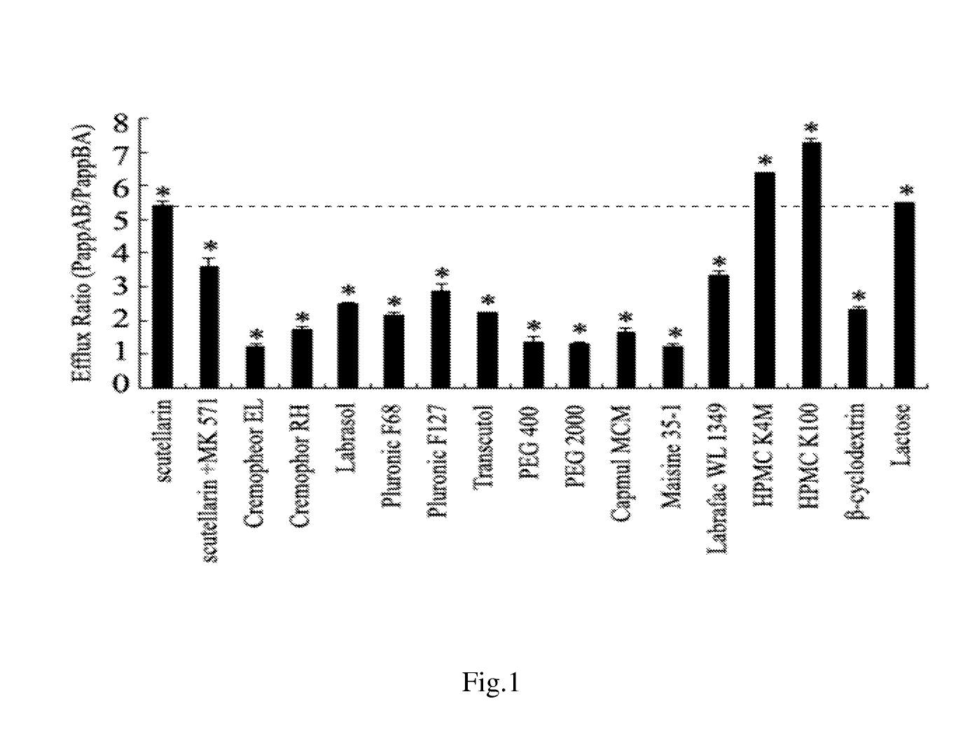

The results of this study were shown in FIG. 1 and Table 3 below. In the Caco-2 permeation analysis, if the calculated efflux ratio of one excipient group was lower than scutellarin standard group, this excipient might possibly be indicated to have the inhibition effect on MRP2.

TABLE 3
Permeability of excipients tested on Caco-2 monolayers
(results in mean ± SD from triplicate experiments).

| | | Papp_{AB} | Papp_{BA} |
|---|---|---|---|
| Excipients | | (10⁻⁶ cm · s⁻¹) | (10⁻⁶ cm · s⁻¹) |
| surfactants | Cremophor ® EL | 4.31 ± 0.24 | 5.21 ± 0.53 |
| | Cremophor ® RH | 2.84 ± 0.37 | 4.93 ± 0.48 |
| | Labrasol ® | 2.25 ± 0.09 | 5.64 ± 0.71 |
| | Pluronic ® F68 | 2.03 ± 0.35 | 4.38 ± 0.13 |
| | Pluronic ® F127 | 1.84 ± 0.09 | 5.31 ± 0.49 |
| oils | Capmul ® MCM | 1.76 ± 0.22 | 2.95 ± 0.19 |
| | Maisine ® 35-1 | 1.04 ± 0.10 | 1.29 ± 0.37 |
| | Labrafac | 1.82 ± 0.15 | 6.11 ± 0.54 |
| | Lipophile ® | | |
| | WL1349 | | |
| co-surfactants | PEG 400 | 1.46 ± 0.08 | 1.96 ± 0.05 |
| | PEG 2000 | 9.91 ± 0.35 | 12.91 ± 1.60 |
| | Transcutol ® | 2.69 ± 0.35 | 5.99 ± 0.52 |
| solid carriers | HPMC K4M | 4.74 ± 0.44 | 30.19 ± 2.33 |
| | HPMC K100 | 6.33 ± 0.34 | 46.10 ± 2.26 |
| | β-cyclodextrin | 4.04 ± 0.33 | 9.33 ± 0.36 |
| | lactose | 1.44 ± 0.24 | 7.90 ± 0.43 |

Combined with its lowest efflux ratio shown in FIG. 1, Cremophor® EL probably affected the activity of MRP2 more than the other four surfactants. For efflux ratios from Caco-2 model in the surfactant group, all of the five surfactants exhibited lower efflux ratios than scutellarin, indicating that they all reduced the efflux values of scutellarin and might thus have the inhibition effect on MRP2. Efflux reduction sequence of the four other surfactants was Cremophor® RH>Pluronic® F68>Labrasol®>Pluronic® F127 (p<0.05).

In the Caco-2 cell monolayers permeation analysis of the oils group, Labrafac Lipophile® WL 1349, Capmul® MCM and Maisine® 35-1 all showed lower efflux ratios than the scutellarin standard group as demonstrated in FIG. 1, so the three oils exerted a reduction effect on scutellarin efflux quantities. Among them, Labrafac Lipophile® WL 1349 had the highest Papp_{AB} value than those of Capmul® MCM and Maisine® 35-1 (p<0.05), while the efflux ratio of Maisine® 35-1 was the lowest comparing with Labrafac Lipophile® WL 1349 (p<0.05). This result suggested that Labrafac Lipophile® WL 1349 may be a better scutellarin absorption enhancer, but not a better MRP2 inhibitor, than the other two tested oils.

In the study of co-surfactants using the Caco-2 cell monolayer, PEG 2000 exhibited the highest permeability from AP to BL direction (p<0.05). As shown in FIG. 1, PEG 400, PEG 2000 and Transcutol® all showed lower efflux ratios than the scutellarin standard group, with PEG 2000 having the lowest efflux ratio (p<0.05). This result indicated that the three co-surfactants could all reduce efflux ratio, and PEG 2000 possibly possess the highest inhibition activity on MRP2 among the other tested co-surfactants.

For solid carriers, Papp_{AB} value was highest in HPMC K100 in Caco-2 cell monolayers (p<0.05) signifying enhanced scutellarin absorption from AP to BL side. As shown in FIG. 1, only β-cyclodextrin showed lower efflux ratio than the scutellarin standard group (p<0.05) among the other tested solid carriers.

### Example 13

In order to investigate the inhibitory potency of excipients against MRP2-mediated transport, membrane vesicles prepared from insect Sf9 cells over-expressing human MRP2 (BD Biosciences) were measured with scutellarin as the probe. Scutellarin replaced the previous usage of E2-17βG as the substrate for detections in the MRP2 transport assay. With some changes in BD protocol of MRP2 vesicles assay, a rapid filtration technique of multiscreen HTS vacuum manifold (Millipore) was used in the analysis. The reaction mixture contained 60 μl vesicles, 2.5 mM GSH, tested excipients, scutellarin and/or MK 571. The mixture was incubated in the buffer (250 mM sucrose, 10 mM MgCl₂, 10 mM Tris/HCl, pH 7.4) at 37° C. for 5 mM, followed by addition of 15 μl of 25 mM MgATP or blank buffer to each well started the test. The mixture was incubated for 4 min and stopped by transferring the membrane vesicles to a filter plate. After washing the filter plate 3 times, the filter paper of each 96 wells was dried and tripled extracted with methanol for the measurement of scutellarin using LC-MS.

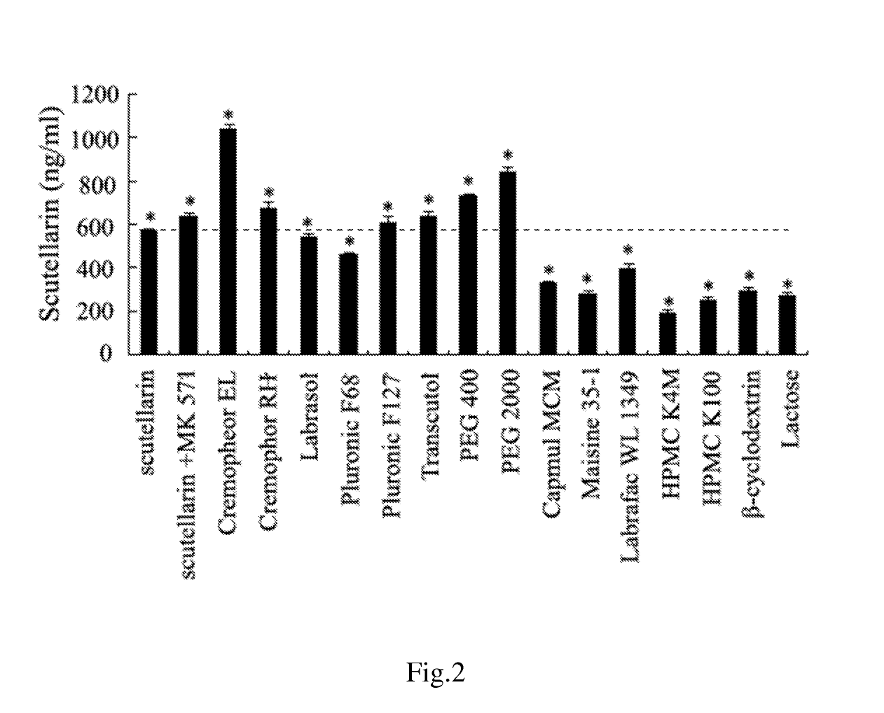

The results of this study were shown in FIG. 2. In the MRP2 membrane-vesicles transport assay, if the detected quantities of scutellarin of one excipient group was higher than scutellarin standard group, this excipient could definitely be verified to have the inhibition ability on MRP2.

In the MRP2 membrane vesicles transport assay of the surfactants group, the sequence of inhibition effects on MRP2 was Cremophor® EL>Cremophor® RH>Pluronic® F127 (p<0.05). However, in this assay, scutellarin concentrations measured in the Labrasol® and Pluronic® F68 groups were not higher than that of the scutellarin standard group and so these two excipients did show any inhibition activity on MRP2.

Summarizing the findings from Examples 12 and 13, Cremophor® EL, Cremophor® RH and Pluronic® F127 could definitely inhibit MRP2, while Labrasol® and Pluronic® F68 did not have the inhibition effect. Their reduction effects on scutellarin efflux may be deduced from their tight junction modulations in Caco-2 cell monolayers but not in the MRP2 membrane vesicles transport assay.

In the study of co-surfactants using the MRP2 membrane vesicles, the inhibition sequence was PEG 2000>PEG 400>Transcutol®, and the concentration of scutellarin detected in PEG 2000 group was also more than those in PEG 400 and Transcutol® (p<0.05). The same results in transport assay verified the sequence deduced from the Caco-2 permeation analysis described in Example 12.

In the MRP2 membrane vesicles transport assay of the oils group, the measured scutellarin concentrations were all lower than the scutellarin standard group. This suggested that although the three oils possessed the efflux reduction ability in the Caco-2 cell model, none of the three oils showed inhibition effect on MRP2 in the transport model. This result might attribute to the differences of physiological environment in the two models, for example, their variations on tight junction existence and changes of osmotic pressure by excipients.

The results in the solid carriers group were similar to the oils group, even β-cyclodextrin showed lower quantities of scutellarin than that of the standard group, suggesting that none of the four solid carriers possessed the inhibition effect on MRP2. Although some of the excipients showed scutellarin efflux reduction ability in the Caco-2 cell permeation assay described in Example 12, none of the excipients in the solid carriers groups and oils group were mechanically proved by MRP2 transport tests to have the specific inhibition effect on MRP2.

### Example 14

Caco-2 cells were cultured for 21 days before they were stable and applied in the permeability tests. These cells were seeded on 24-well insert filters with a density of 1×10⁵ cells/well. Caco-2 cell monolayers have been evaluated by analyzing its integrity, impermeability and differentiation respectively.

The transport study on the Caco-2 cell monolayers was to evaluate apparent permeability coefficient (Papp) in two directions of the cell monolayers. The Papp_{ab} was the permeability value from the apical (AP) to the basolateral (BL) side and Papp_{ba} was in the direction from BL to AP side. The permeability tests were carried out at a condition of 37° C. with 5% CO₂ after equilibration. The equilibration was the addition of 400 μl HBSS (pH 7.4) in AP side and 600 μl HBSS in BL side for 30 min after washing the cultured cells three times with HBSS buffer in 37° C.

In the synergistic-effect analysis, 100 μM scutellarin was also added in each test as the analytical probe. The transport experiments of solo Cremophor® EL and the mixed nine excipients samples were analyzed following similar procedures in the dose-dependent assay. The comparative sample of Cremophor® EL of 400 or 600 μl (Papp_{ab} or Papp_{ba}) was added in the donor chambers. The volume added for each excipient in the two samples of the nine mixed samples was 200 or 300 μl (Papp_{ab} or Papp_{ba}) in the donor chambers. The experiments were performed at 37° C. with 5% CO₂ for 30, 60 and 90 min. The efflux ratios were calculated by dividing the values of Papp_{ba} with Papp_{ab}.

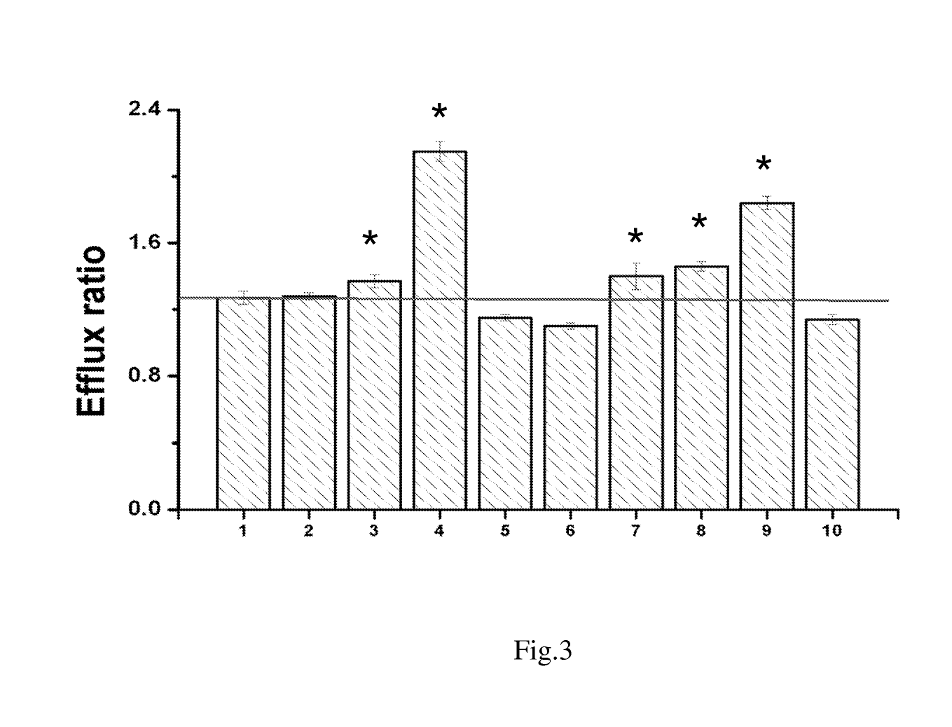

The results of efflux ratio were shown in FIG. 3. In the Caco-2 cell monolayer model, three groups, namely Cremophor® EL+ Pluronic® F127, Cremophor® EL+ PEG 2000 and Cremophor® EL+ β-cyclodextrin, showed a joint enhancement effect in which the resulting effects thereof were better than the effect by Cremophor® EL alone. These three groups were shown to reduce efflux ratio at the percentage of 11.63%, 16.58% and 12.91% respectively. The results demonstrated that the surfactant (Pluronic® F127), co-surfactant (PEG 2000) and solid carrier (β-cyclodextrin) had the positive synergistic effect in reducing efflux of scutellarin, and it may indicate that these three combined-excipient groups could have the synergistic inhibition effect on MRP2.

### Example 15

In the synergistic-effect analysis, scutellarin at 100 μM was added to each sample in the MRP2 transport model as the assay probe. The experiment was divided into two assay groups: the comparative group and the mixed sample group. The comparative group was composed of scutellarin and 100 μg/ml Cremophor® EL. The mixed excipient groups in the MRP2 transport assay included five pairs of excipients: Cremophor® EL+ Cremophor® RH, Cremophor® EL+ Pluronic® F127, Cremophor® EL+ PEG 2000, Cremophor® EL+ PEG 400, and Cremophor® EL+ Transcutol®. Each excipient in the mixed excipient group had the concentration of 100 μg/ml. 1 mg/ml sf9 over-expressed MRP2 vesicles was used in the MRP2 transport inhibition assay. Upon preparations of the tested reagents and samples, the inhibition reactions were carried out with the addition of 20 μl MRP2 vesicles and 100 μl samples from different groups respectively. The tests were conducted at 37° C. for 40 min. With the addition of stop solutions of cold HBSS buffer, the transport solutions in the 96-well transparent plates were transferred to 96-well filtration plates (0.7 μm pore size). Upon vacuum filtration and washing with assay buffer, the diluted solutions were collected with the existence of ATP or with AMP. On comparing with the radioactive method in previous studies, absorbance at 463 nm was chosen to be detected in micro-plate readers for the presence of MRP2 substrate scutellarin.

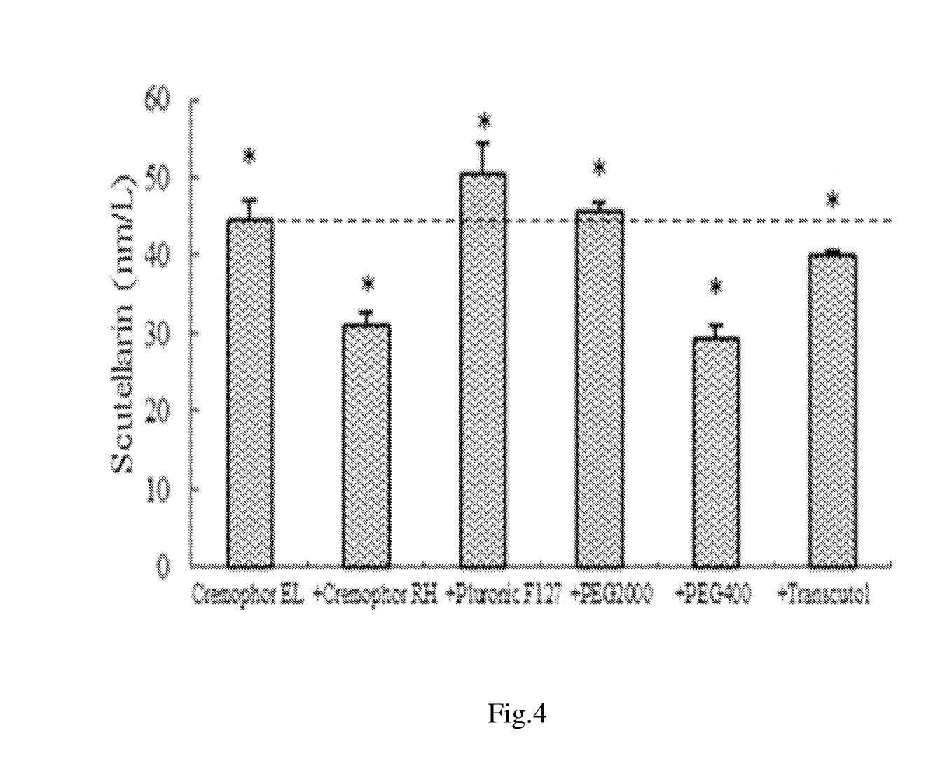

The results of this study were shown in FIG. 4 that illustrated the synergistic inhibition effect between Cremophor® EL and the five excipients of Cremophor® RH, Pluronic® F127, PEG 2000, PEG 400 and Transcutol®. From the listed transported quantities of scutellarin, Pluronic® F127 and PEG 2000 were shown to increase the inhibition effect on MRP2 at 13.76 and 1.23% respectively. On comparing with the results in Caco-2 cell model, the data in FIG. 4 showed that both the Cremophor® EL+ 690 Pluronic® F127 group and the Cremophor® EL+ PEG 2000 could synergistically decrease efflux of scutellarin together with Cremophor® EL, indicating that these two pairs of excipients (i.e. Cremophor® EL+ Pluronic® F127 and Cremophor® EL+ PEG 2000) inhibited the MRP2 protein.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

What is claimed is:

1. A pharmaceutical composition for treating cardiovascular and cerebrovascular diseases comprising scutellarin and an adjuvant selected from a group consisting of a co-surfactant, a surfactant, an oil, a solid carrier, and any combinations thereof.

2. The pharmaceutical composition of claim 1 wherein said co-surfactant is selected from a group consisting of diethylene glycol monoethylether, polyethylene glycol (PEG), and any combinations thereof.

3. The pharmaceutical composition of claim 1 wherein said surfactant is selected from a group consisting of polyoxyethyleneglycerol triricinoleate 35 castor oil, polyoxyethylene hydrogenated castor oil, poloxamer 407, poloxamer 188, gaprylocaproyl macrogolglycerides, and any combinations thereof.

4. The pharmaceutical composition of claim 1 wherein said oil is selected from a group consisting of glyceryl monolinoleate, medium chain triglycerides, C8/C10 mono-/di-glycerides, and any combinations thereof.

5. The pharmaceutical composition of claim 1 wherein said solid carrier is β-cyclodextrin.

6. A pharmaceutical composition for treating cardiovascular and cerebrovascular diseases comprising scutellarin and polyoxyethyleneglycerol triricinoleate 35 castor oil.

7. The pharmaceutical composition of claim 6 further comprises poloxamer 407, PEG 2000, and any combinations thereof.

8. A method of treating cardiovascular and cerebrovascular diseases in a human patient comprising administering to the patient a pharmaceutical composition comprising scutellarin, polyoxyethyleneglycerol triricinoleate 35 castor oil, and an adjuvant wherein said adjuvant exhibits a synergistic effect with polyoxyethyleneglycerol triricinoleate 35 castor oil in treating cardiovascular and cerebrovascular diseases.

9. The method of claim 8 wherein said adjuvant is selected from a group consisting of poloxamer 407 and PEG 2000.

10. A composition for inhibiting multidrug resistance-associated protein-2 (MRP2) comprising scutellarin and an adjuvant selected from a group consisting of a co-surfactant, a surfactant, and any combinations thereof.

11. The composition of claim 10 wherein said co-surfactant is said co-surfactant is selected from a group consisting of diethylene glycol monoethylether, polyethylene glycol (PEG), and any combinations thereof.

12. The composition of claim 10 wherein said surfactant is selected from a group consisting of polyoxyethyleneglycerol triricinoleate 35 castor oil, polyoxyethylene hydrogenated castor oil, poloxamer 407, poloxamer 188, gaprylocaproyl macrogolglycerides, and any combinations thereof.
